(19) United States
(12) Patent Application Publication
(10) Pub. No.: US 2005/0048005 A1
(43) Pub. Date: Mar. 3, 2005

(54) Antimicrobial compositions for dental applications

(76) Inventor: Richard F. Stockel, Bridgewater, NJ (US)

Correspondence Address: RICHARD F. STOCKEL, 475 ROLLING HILLS RD., BRIDGEWATER, NJ, 08807, US

(21) Appl. No.: 10/647,752

(22) Filed: Aug. 26, 2003

(51) Int. Cl. A61K 7/16; A61C 5/00

(52) U.S. Cl. 424/49; 433/217.1

(57) ABSTRACT

This invention relates generally to antiplaque/gingivitis mouth rinses conductive to oral hygiene, and more particularly to a mouth rinse whose formulation includes new compositions whose compositions include a metathesis or acid-base reaction of two well know anti-bacterial agents, or combinations thereof. The novel compositions of this invention can also be used in dentifrice, additive for dental floss, and antimicrobial coatings for sealing fissures, and the like, and for long term protection against caries.

### INTRODUCTION

The prevention and control of periodontal diseases is important, not only to maintain a healthy and functional natural dentition, but also to reduce the risks of systemic complications.

It is known that bacteria and their products initiate and perpetuate the process of tissue destruction; thus any preventive care should be focused on the bacteria to control periodontal diseases.

Since mechanical measures are clearly failing to maintain periodontal health, a strong emphasis has been placed on providing therapeutic agents that will provide better levels of bacterial control. Since gingivitis is a rather non-specific infection, clearly a requirement for an anti plaque agents to improve gingival health should have a broad spectrum of antibacterial activity and be substantive in the mouth (teeth and tissue) for a prolonged period of time.

### DETAILS OF THE INVENTION

This invention relates to new biocidal complexes prepared by metathesis synthesis involving either a monomeric or polymeric cationic biocide reacted with the anionic form of a biocide of a monomeric or polymeric biocide which are useful for a variety of dental applications, e.g., mouthwash, dentifrice, dental floss coating and as a dental coating or sealants to protect teeth. A second synthetic route is sometimes possible, and it involves the reaction of an acid with a base to yield a salt like product. This is feasible when the conjugate base (free base) of the cation is reacted with the conjugate acid of the anion provided the pkb and/or pka are sufficiently either a strong base or strong acid. These complexes tend to have low water solubility therefore for many, but not all applications it is necessary to prepare emulsions or microemulsions to obtain a stable aqueous solution. These complexes are very effective biocides against a variety of bacteria, fungi and other microorganisms.

Individually, the biocides of this invention are well known in the published literature, however the complexes of this invention are quite unique, novel and represent new biocidal compositions, emulsions, and microemulsions, thereof.

In accordance with this invention, the effectiveness of individual biologically active compounds can be enhanced by the formation of these complexes as described by this invention. Thus the combination of a bioactive cation with a bioactive anion improves the overall biological activity.

This invention has other important safety and toxicity implications because the resulting complex can be composed of either EPA or FDA approved materials.

Another advantage involves the green chemistry used in synthesizing these compositions. Fortunately, the metathesis reaction can be carried out in a totally aqueous medium. The by-product of this reaction is a salt, which does not represent any serious environmental problem for disposal. In fact, many salts can be recycled for other uses. If the acid-base reaction is appropriate, then there is no by-product at all.

While the literature is replete with many patents and articles concerning the individual components of this invention, there is scarce mention of preparing the complexes of this invention. For example, WO 97/25085 describes the combination (admixture) of chlorhexidine with triclosan to contribute antimicrobial activity when applied to medical devices and the like. The inventors do not anticipate our technology, because no mention is made about a chemical reaction between these two biocides, nor does the method they use to apply these biocides allow the formation of a complex.

U.S. Pat. No. 5,575,993 discloses compositions of polyionenes with anionic biological species. However, my invention is not anticipated by 993', since the two are significantly different from each other. These differences are clearly delineated in 993' whereby only part of the polyionene anion is replaced by a bioactive species, from about 0.005 to about 0.33 or 0.50 degree of substitution depending on the specific polyionene used. All of the resulting compositions are very soluble in water, unlike the compositions of my invention, prior to solubilization with the assistance of surfactants and cosolvents.

Chlorhexidine reacted with anionic polymers like algin or or carboxymethylcellulose is taught in U.S. Pat. No. 4,980,150. The purpose of this invention is to prepare a water insoluble salt which has no biocidal synergy, and its' sole purpose is to form a granulated powder to be used as a dentifrice.

U.S. Pat. No. 6,500,466 teaches the preparation of chlorhexidine sugar acids or lactones of sugars. The resulting compositions have exceptional storage stability. No evidence is provided concerning improved biocidal activity.

Other examples of admixtures can be found in EP 0843,002 A2 and U.S. Pat. No. 6,106,505. The former patent describes a detergent composition containing cationic germicides like benzalkonium or chlorhexidine salts combined with triclosan. In contrast the latter patent teaches the use of the free base, chlorhexidine with triclosan has synergist antimicrobial properties and it is useful for coating medical devices.

The publication, Eur.J. Oral Sci. 1988, 106: pp 571-575, discloses the effect of a mixture of chlorhexidine salt-thymol containing varnish on reducing prostaglandin E₂ levels in gingival crevicular fluid.

Another patent U.S. Pat. No. 6,440,395B1 teaches the use of cetyl pyridinium chloride and triclosan as an admixture, solubilized with surfactants resulting in a anti-plaque mouthwash.

The invention will be illustrated by the following examples, which, it will be understood, are not intended to be limiting, but merely illustrative.

### List of Specific Bioactive Cationic Agents

The following monomeric and polymeric bioactive cationic agents are illustrative of this invention. They by no means represent all possible cationic biocides, but instead are examples of the broad array available to a practitioner who wishes to carry out the scope of this invention.

Examples:
Polyhexamethylene biguanide hydrochloride salt
Polyhexamethylene guanidine hydrochloride salt
Dimethyldidecyl ammonium chloride
Benzalkonium chloride
Benzethonium chloride
Chlorhexidine salts
Polyionenes, e.g., Poly (dimethyl butenyl ammonium chloride) alpha, omega-bis (triethanol-ammonium chloride and poly (oxyethylene (dimethylimino) ethylene (dimethylimino) ethylene dichloride
Dequalinium chloride
Polyquatemium 2
Hexetidine
Octenidine
D,L-pyrrolidone carboxylic acid salt of N^{α}-cocoyl-L-argine ethyl ether (CAE)
Sanguinarine salts
Antibiotics containing amine salt, e.g., tetracycline, doxycycline or minocycline
Cetyl pyridinium chloride
Tetrakis (hydroxy methyl) phosphonium sulfate
Gemini quats, e.g.,—ethanediyl-α, w-bis (dodecyldimethyl) ammonium halide
Quaternary ammonium dendrimeric biocides (U.S. Pat. No. 6,440,405)
Long chain sulfonium salts
Long chain phosphonium salts
Delmopinol salts
Alexidine

It is understood that these cationic antimicrobial agents can be other salts besides the hydrochloride. Some examples are hydroxy carboxylic acids, amino acids, sulfonates, and phosphates to name just a few examples. One skilled in organic chemistry could find other suitable substitutes.

The specific biocides described are illustrative of this invention, but do not represent a complete inventory of all the possible combinations possible. Anyone skilled in the art of chemistry and biology can conceptualize other modifications. In particular, some of the polymeric species useful for carrying out this invention could be further modified by varying the repeating units or by end capping. U.S. Pat. Nos. 4,891,423 and 5,741,886 are examples of further enhancing the antimicrobial activities of phmb. Other such examples for different polymeric systems also exit.

### List of Specific Bioactive Anionic Agents

The following monomeric and polymeric bioactive anions represent a partial list of actives, which can be utilized in this invention. Knowledgeable persons familiar with biocides can conjure other possible anionic substitutes. In keeping with the spirit this of this invention, the list below is illustrative as working examples to achieve very broad antimicrobial activity for a variety of applications.
Sodium hydroxymethyl glycinate
Sodium salicylanilide
Sodium stearate
Thymol
Eugenol
Hinokitiol and substituted tropolone
Sodium undecylenic acid
Sodium ortho-phenylphenol
Sodium triclosan
Sodium polyphosphate
Poly anionic compositions like polydivinyl ether-maleic anhydride alternating copolymer
Anionic dendrimers (U.S. Pat. No. 6,464,971)
Chitosan derivatives having carboxylate, sulfate, sulfonate, phosphonate or phosphate anionic functional groups present in the molecule
EDTA and derivatives having carboxylate anions
1-hydroxy ethane-1, 1-diphosponic acid
Nitrilotris (methylenephosphonic acid)
Ethylenediaminetetrakis (methylene-phosphonic acid)
Mono or di alkyl phosphates or mixtures thereof
Aminophosphonic acids
Antibiotics containing carboxylic acids, e.g., mupirocin

### General Synthesis

Metathesis Procedure

The formation of the candidate molecules can be synthesized by straight forward metathesis reactions carried out in aqueous solutions, or aqueous alcohol mixtures.

These bioactive molecules are produced using the ultimate green chemistry approach. Water is the solvent of choice, by-products are harmless salts and yields are excellent to quantitative.

The appropriate cationic moiety is reacted with the desired anionic moiety in water. The concentration of reactants can vary from 20 to about 60 wt. % of the total solution. The reaction takes place at room temperature, and is generally completed within one hour.

The final product is readily removed by decantation of the solvent and isolation of the solid product and generally can be used as is for certain applications.

Acid-Base Formation of the Complexes

This well known facile reaction can be utilized in some cases by the reaction of a conjugate base (free base) of a biocidal cation with the conjugate acid (protonated) of the biocidal anion. This can be represented by the following example.

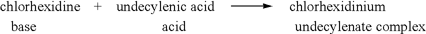

In order for the acid-base process to work the acid component must have a transferable proton (pka) to a basic (pkb) molecule. The reaction is usually conducted in refluxing alcohol (C1-C4), or aqueous alcoholic solutions.

The acid-base reaction is particularly advantageous for the formation of a bioactive azole compounds with biocides that have a protonic hydrogen capable to transfer to a base nitrogen in a azole molecule. This represents a classical acid-base synthetic process. The family of azoles are either imidazole or triazole derivatives. If the azole can be protonated, then it can be subsequently reacted with a anionic monomer or polymer biocide, illustrating a metathesis reaction.

### General Method for the Formation of Emulsions/Microemulsions for the Complexes of this Invention

The complex is dissolved in the minimum amount of a solvent with the appropriate Hildebrand solubility parameter. The solubility parameter is a numerical value that indicates the relative solvency behavior of a specific solvent. Hildebrand solubility parameters from about 8.5 to about 22.0 are suitable for solubilization of the complexes of this invention.

Depending on the ionic/covalent bonding energies of these compositions, the correct solvent for solubilization will be on the low side, if the bonding has more covalency, and if the bonding is more ionic, then the proper solvent will have a much higher value.

Combinations of solvents are also useful in preparing emulsions or microemulsions.

Next, an amphoteric or non-ionic is added to the dissolved complex. Combinations of the above type surfactants can also be utilized. Certain cationic surfactants also are applicable. However, highly negative anionic surfactants are not very functional.

The complex-solvent-surfactant is then diluted with water to the active concentration required for the particular application to form an emulsion or microemulsion depending on the micellar size and choice of solvents/cosolvents.

### Surfactants

Mouth Rinse Application

Expertimently, it has been determined that the preferred surfactants, which form microemulsions (cosolvent is added) or emulsions with the complexes of this invention, are by and large, either amphoteric or non-ionic types, or combinations thereof. Highly charged anionic surfactants have the potential to reduce the overall bioactivity of these complexes by causing some degree of precipitation, thereby lessening its effectiveness.

It was also found that cationic phospholipids, usually in combination with non-ionic and/or amphoteric surfactants have been found to be effective.

Surfactants that carry a positive charge in strongly acidic media carry a negative charge in strongly basic media, and form zwitterionic species at intermediate pH's are amphoteric. The preferred pH range for stability and effectiveness is from about 5.0 to about 9.0. Under this pH range, the amphoteric surfactant is mostly or fully in the zwitter (neutral) form, thereby negating any dilution of bioactivity of the compositions of this invention, provided it's usage is in the preferred concentration range of about 0.25 to about 4.0 wt. % based on the actives.

It has been observed that amphoteric amidobetaine surfactants are particularly preferred in solubilizing the complexes of this invention to produce clear aqueous or aqueous-alcohol mouth rinse solutions.

One aspect of this invention therefore provides a mouthwash composition comprising a biocidal complex, and effective amount of a non-ionic, amphoteric, or cationic surfactant, or combination thereof, and other incipients found in a mouthwash like chelating agents, organic carboxylic acids, flavors, sweeteners and optionally alcohol.

An important ingredient in a mouthwash is the surfactant(s). The following surfactants have been found to perform effectively in forming microemulsions or semi-transparent emulsions with the antimicrobial agents of this invention.

These include amphoteric amido betaines, non-ionic polyethoxylated sorbital esters, polycondensates of ethylene oxide-propylene oxides (polyxamers), polyethoxylated hydrogenated castor oils, and certain cationic phospholipids.

Suitable examples of amidobetaines include cocoamidoethylbetaine, cocoamidopropyl betaines or mixtures thereof. Alternative amphoteric surfactants include long chain imidazole derivatives such as the product marketed under the trade name “Miranol C2M” by Rhodia and long chain alkyl betaines, such as the product marketed under the tradename “Empigen BB” by Huntsman Corporation, and mixtures thereof.

Suitable nonionic surfactants include polyethoxylated sorbital esters, in particular polyethoxylated sorbital monoesters, for instance PEG (40) sorbitan di-isostearate, and the products marketed under the trade name “Tween” by ICI; polycondensates of ethylene oxide and propylene oxide (poloxamers), for instance the products marketed under the trade name “Pluronic” by BASF; condensates of propylene glycol; polyethoxylated hydrogenated castor oil like the “Cremophors” by BASF and sorbitan fatty esters by ICI. Other effective non-ionic surfactants include the polyalkyl (C₈-C₁₈) glucosides

Suitable cationic surfactants include D,L-2-pyrrolidone-5-carboxylic acid salt of ethyl-N-cocoyl-L-arginate (CAE), marketed by Ajinomoto, and cocamidopropyl (PTC), lauramidopropyl PG dimonium chloride phosphates and the like sold by Uniqema. Two of the above cationic surfactants, CAE and PTC having significant antimicrobial activity can be used as the positive cation of the binary cation-anion bioactive complexes of this invention.

Experimentally, it has been found that the amount of surfactant(s) either individually or in combination ranging from 0.25 to about 4.0 wt % based on the antimicrobial complex.

Generally, other incipients are normally added to a mouthwash final formulation. These include water or aqueous ethanol, and optionally a further liquid such as glycerin or propylene glycol. Such mouthwashes may also contain humectants, thickening agents, flavoring agents, sweetening agents, coloring agents and preservatives.

### Examples—Solubilization of Complexes Concentrates Dilutable with Water

1. phmb triclosante
20 g active
150 g ethanol
0.8 g Tego Betaine Z (real)

2. chlorhexidinium di-triclosate
20 g active
150 g ethanol
0.3 g Tween 20/0.5 g Tego Betaine ZF

3. chlorhexidium di-stearate
20 g active
200 g isopropanol
0.3 g Tween 20/0.5 g Tego Betaine ZF

4. phmb-triclosate
20 g active
200 g ethanol
0.3 g Tween 20/0.5 g Tego Betaine ZF

5. phmb-thymol
20 g active
200 g ethanol
0.3 g Tween 20/0.5 g Tego Betaine ZF

6. CAE-triclosate
20 g active
200 g ethanol
0.8 g Cremaphor CO-40

### Microbiological Tests

The bacteriostatic activity of several complexes was investigated by testing at 0.1 wt. % using Oxoid No. 2 nutrient broth and inoculating the broth with 1 ml of a 24 hour broth culture of the test organisms. After incubation at the optimum growth temperature of the organism for 48 hours.

The organisms tested were:
Staphylococcus aureous (gram positive)
Pseudomonas aeruginosa (gram negative)
Escherichia coli (gram negative)

All six complexes were tested and found to be bacteriostatic at 0.1 wt. % against the above 3 organisms. These complexes were the only one studied using this test.

### Dentifrice

The binary biocidal complexes of this invention are useful in the formulation of a dentifrice for reducing the formation of plaque, thus inhibiting periodontal diseases.

Dental plaque is a soft deposit, which forms on teeth and is comprised of an accumulation of bacteria and bacterial by-products. Plaque adheres tenaciously at the points of irregularity or discontinuity e.g. on rough calculus surfaces, at the gum line and the like. Besides being unsightly, plaque is implicated in the occurrence of gingivitis and other forms of periodontal disease.

Historically, chlorhexidine and triclosan are perhaps the best-known antiplaque agents, which have been investigated by numerous scientists resulting in commercial products.

Chlorhexidine is acknowledged to be more effective then triclosan, however the former chemical causes noticeable staining in the majority of users. This unsightly stain can only be removed by a dental office visit where it is mechanically removed. Attempts to include abrasives, anionic surfactants to reduce staining is hampered due to the incompatible of the bis-biguanide chlorhexidine, and tend to diminish the bioavailability of agent as well.

The cationic-anionic dual biocide complexes of this invention can readily be formulated into a toothpaste having effective antiplaque properties and little or no staining, which typically comes from the cationic moiety, e.g., chlorhexidine, cetyl pyridinium chloride, quats, etc. which exist in a water soluble form in the mouth cavity when using water soluble cationic biocides.

The biocidal complexes of this invention have limited water solubility and probably operate as a slow release reservoir of the combined, cationic-anionic, complex. This is one possible explanation, not necessarily the only one.

The dentifrice compositions useful in the present invention, in which the biocidal complexes are present, comprise from about 0.01 to about 5.0% by weight of the complex.

Incipients normally found in dentifrice are surfactants similar to those discussed in the mouthwash section of this application including humectants, thickeners, foaming surfactants and abrasives. Favoring, sweetening and coloring agents are also frequently used.

Dentifrice employing the antiplaque compositions of this invention can be formulated using the following formulation outlined in Table 1.

TABLE 1

| Ingredients | % by Weight |
|---|---|
| Glycerine | 8 |
| Sodium carboxymethyl cellulose | 1.5 |
| Sorbital | 38 |
| Sodium monofluorophosphate | 0.8 |
| Sacchrarin, sodium | 1.0 |
| Sodium dihydrogenm phosphate | 0.05 |
| Sodium monohydrogen phosphate | 0.25 |
| Silica, hydrated | 15.0 |
| Titanium dioxide | 0.25 |
| Flavor | 2.0 |
| Antiplaque agent of this invention | 0.5 |
| FD & C dye | 0.0003 |
| Deionized water | Q.S. to 100 |

### Dental Floss

A third important dental use for the biocidal compositions of this invention involves germicidal dental floss.

It is well known that periodontal disease affects the supporting tissues of teeth, bone, periodontal ligament, cementum and gingival. The reason for periodontal disease is bacterial plaque accumulation on the tooth surfaces. The most difficult areas to reach by brushing or mouthwash for proper oral hygiene are the interproximal surfaces of the teeth. These areas are best cleaned with the aid of dental floss. The various types of dental floss used in the prior art mostly effect only a mechanical cleaning of the interproximal tooth areas.

Dental flosses have long been used effectively to clean the spaces between the teeth and under the gum margin. To increase the effectiveness of the floss, fluoride or bactercides can be added in the bulk or as a coating. By the proper use of dental floss, it has been found to be effective in inhibiting tooth decay and gum diseases.

Dental floss can be made of natural or synthetic fibers, e.g., teflon, nylon, polypropylene and it can contain a wax to reduce function.

The dual biocidal cationic-anionic complexes of this invention can be either dispersed or dissolved in the commonly used binders e.g., wax, hydrophilic polymers, polyalkylene glycols, and the like, to coat the dental floss material.

Certain compositions, where the anionic biocidal portion of the complex is a long chain carboxylate can function as a anti-friction agent in addition to the complex in general having antimicrobial activity.

The complexes would slowly erode off the dental floss and deposit on the tooth structure and oral cavity when used to clear teeth. The following example describes how a non-wax commercial dental floss can be coated with a chlorhexidine-triclosan complex for use as a germicidal dental floss. The biocidal complexes of this invention should be present from about 0.10 to about 10.0 wt. %.

### Example: A 5 wt % Biocidal Coated Dental Floss

A. To a 5 g sample of a chlorhexidine-triclosan complex was added 60 g of PEG 3350, 30 g PEG 1000, and 5 g glycerin to dissolve the complex by stirring and gentle heating. To this warm solution a commercial non-wax dental floss was coated to give the desired treated dental floss.

B. To a 5 g sample of a chlorhexidine-stearate complex was added 60 g of PEG 3350, 30 g PEG 1000, and 5 g glycerin to dissolve the complex by stirring and gentle heating. To this warm solution a commercial non-wax dental floss was coated to give a wax like antimicrobial dental floss.

### Coating for Caries Prevention

This invention also concerns the use of these dual biocidal complexes with long term activity, comprising a physiologically acceptable coating base and dissolved therein the antimicrobial complex. The resulting coating can be painted onto teeth to afford long term protection against caries.

The complexes, including chlorhexidine-triclosan, chlorhexidine-thymol, phmb-triclosan, and phmb-thymol were dissolved in a suitable safe solvent like ethanol, and a biocompatible polymer.

Said biocompatible polymers can be polypropylene glycols, polyvinyl acetate-c-vinyl alcohol, or poly 2-hydroxyethyl methacrylate. Other polymers can be utilized, which have slight water solubility and is compatible with the complex-solvent, and has a very low toxity.

### Example of a typical Formulation

| | |
|---|---|
| 5% w/w | chlorhexidine-triclosan complex |
| 20% w/w | 60% vinyl acetate-40% vinyl alcohol/copolymer |
| 75% w/w | ethanol |

This resulted in a thin-liquid low viscosity coating

The antimicrobial complexes of this invention are used for teeth coatings in effective concentrations of about 1.0 to about 15.0 wt. %.

1. The method for preparing a antimicrobial complex useful as a mouthwash, dentifrice, coating for a dental floss, or a protective coating for teeth by a metathesis reaction between a cationic biocidal monomer or polymer with an anionic biocidal monomer or polymer.

2. The method for preparing a antimicrobial complex useful as a mouthwash, dentifrice, coating for a dental floss, or a protective coating for teeth by an acid-base reaction between a biocidal free base and a biocidal organic compound capable of donating a proton to the free base.

3. A method as defined in claim 1 wherein the cationic monomeric biocide has an amidine, guanidine, biguanide, a protonated tertiary amine antibiotic or a quaternary functionality.

4. A method as defined in claim 3 wherein the cationic monomeric biocide is chlorhexidine salt, cetyl pyridium halide, benzalkonium halide, sangiunarine halide, D,L-pyrrolidone carboxylic acid salt of Nα-cocoyl-L-argine ethyl ether, domiphen bromide, ethanediyl-α,w-bis (dodecyldimethyl) ammonium halide, delmopinol halide, tetracycline hydrochloride, doxycycline hydrochloride or minocycline hydrochloride.

5. A method as defined in claim 1 wherein the cationic polymeric biocide has a amidine, guanidine, biguanide, quaternary functionality in the backbone, or side chain, or contained in dendrimers.

6. A method as defined in claim 5 wherein the cationic polymeric biocide is Polyhexamethylene guanidine, Polyhexamethylene biguanide, or a quaternary dendrimer.

7. A method as defined in claim 1 wherein the anionic monomeric biocide has phenolic, carboxylate, tropolone, and organophosphate, organophosphonate, or inorganic oxyphosphorus functionalities.

8. A method as defined in claim 7 wherein the anionic monomeric biocide is triclosan, o-phenylphenol, thymol, eugenol, 4-isopropyl-tropalone, unidecylenic acid, mupirocin, mono or di alkyl phosphates, ethylenediaminetetrakis (methylene-phosphonic acid), phosphate or pyrophospate.

9. A method as defined in claim 2 wherein the biocidal base is a teriary amine such as sanguinarine, tetracycline, doxycycline, minocydine or delmopinol.

10. A method as defined in claim 2 whreein the biocidal acid is unidecylenic, stearic, mupirocin, or salicyclic carboxylic acids.

11. A method for the preparation of a mouthwash comprising:
a.) from about 0.01 to about 1.5 wt. % of a biocidal complex as described in claim 2;
b.) from about 0.25 to about 4.0 wt. % based on actives, and;
c.) optionally containing up to 20 wt. % ethanol;
d.) diluted to 100 wt. % with water

12. A method for the preparation of a mouthwash comprising:
a.) from about 0.01 to about 1.5 wt. % of diocidal complex as described in claim 1;
b.) from about 0.25 to about 4.0 wt. % of a cationic, non-ionic or a betaines surfactant based on actives, and;
c.) optionally containing up to 20 wt. % ethanol;
d.) diluted to 100 wt. % with water

13. A method as defined in claim 9 wherein the surfactants are polyalkoxylated sorbital long chain hydrocarbon esters as the non-ionic surfactants, long clain hydrocarbon amidopropyl-betaine as the ampholeric type surfactants, phospholipids as the cationic surfactants, or combinations thereof.

14. A method as defined in claim 10 wherein the surfactants are polyalkaxylated sorbital long chain esters as the non-ionic surfactants, long chain hydrocarbon amidopropyl betaines as the amphoteric surfactants, phospholipids as the cationic surfactants or combinations thereof.

15. A method to prepare a dental floss wherein the anti-plaque complex as described in claim 1 is present in bulk or as a coating from about 0.10 to about 10.0 wt. %.

16. A method to prepare a dental floss wherein the anti-plaque complex as described in claim 2 is present in bulk or as a coating from about 0.01 to about 10.0 wt. %.

17. A method a preparing a dentifrice comprising a biocidal complex as described in claim 1 in amounts of about 0.01 to about 5.00 wt. %, a solubilizing solvent in amounts of about 5.0 to about 20.0 wt. % a thickening polymer and a humectant in amounts of about 0.2 to about 10.0 wt. %, then adding a non-ionic, amphoteric, cationic or combinations thereof to form a gel.

18. A method of preparing a dentifrice comprising a biocidal complex as described in claim 2 in amounts of about 0.01 to about 5.00 wt. %, a solubilizing solvent in amounts of about 5.0 to about 20 wt. %, a thickening polymer and a humectant in amounts of about 0.2 to about 10.0 wt. %, then adding a non-ionic, amphoteric, cationic or combinations thereof to form a gel.

19. Method of preparing a dental coating using the biocidal complexes of claim 1 useful to protect teeth against gingivitis, caries and the build up to plaque, used in concentrations of about 1.0 to about 15.0 wt. %.

20. Method of preparing a dental coating using the biocidal complexes of claim 2, useful to protect teeth against gingivitis, caries and the build up to plaque, used in concentrations of about 1.0 to about 15.0 wt. %.
